(19) United States
(12) Patent Application Publication
(10) Pub. No.: US 2006/0189958 A1
(43) Pub. Date: Aug. 24, 2006

(54) Inverted cannula for use in reconstituting dry material in syringes

(76) Inventors: James D. Talton, Alachua, FL (US); James F. Kirk, Del Mar, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/358,348

(22) Filed: Feb. 21, 2006

Related U.S. Application Data
(60) Provisional application No. 60/593,875, filed on Feb. 21, 2005.

(51) Int. Cl. A61B 19/00

(52) U.S. Cl. 604/416

(57) ABSTRACT

The present disclosure relates to a device (such as, e.g., a syringe) for reconstituting a material to be reconstituted, comprising (a) a first structure having at least one first inner portion configured to receive at least a portion of a material to be reconstituted, comprising a first proximal opening and a first distal opening, wherein the first distal opening has a first mating structure, and a first movable member selectively inserted into said first inner portion to urge out the material to be reconstituted, (b) a second structure having at least one second inner portion configured to receive at least a portion of a reconstituting material, comprising a second proximal opening and a second distal opening, wherein the second distal opening has a second mating structure, and a second movable member selectively inserted into said second inner portion to urge out the reconstituting material, and (c) a connector device, comprising a first mating portion configured to mate with the first mating structure, a second mating portion configured to mate with the second mating structure, and a lumen extending between the first and second mating portions and configured to permit communication between the materials in said first and second structures. The disclosure further relates to methods for reconstituting a material to be reconstituted.

This Application claims the benefit of U.S. Provisional Application No. 60/593,875, filed Feb. 21, 2006, the disclosure of which is incorporated herein by reference.

### FIELD OF THE INVENTION

The present disclosure relates generally to devices and methods for reconstituting a material to be reconstituted, such as, e.g., a dry powder, particulate, or fibrous material. In one embodiment, the invention relates to a method and device to facilitate in reconstituting dry materials in syringes by introducing a reconstituting fluid to that end of such syringes farthest from the mouth of such syringes, allowing easy mixing and expelling of the material.

### BACKGROUND

This disclosure relates to a device to aid in reconstituting dry powders, particulate materials, and/or fibrous materials in devices such as, e.g., syringes. Many biologic products store well if reduced to a dry form, such as, e.g., antibiotics, protein powders, and some drugs. However, reconstituting these materials so that these can be injected with a syringe often requires not only the addition of a reconstituting fluid such as sterile water, saline, whole blood, or blood components (e.g., serum), but also mixing of the dry material and the reconstituting fluid. During such mixing it is often desirable to exclude as much air as possible, and if dispersion of the liquid is difficult a homogenous mixture may not be possible.

It is conventional to store drugs, vaccines, medicaments, and solutions in a sealed vial or other container for later use. Drugs, vaccines, medicaments, and solutions may be stored in a dry or powdered form to increase the shelf life and reduce inventory space. Such dry or powdered materials are typically stored in a conventional sealed vial having a puncturable closure, such as an elastomeric stopper, and reconstituted in liquid form for later use, such as administration to a patient, by adding a diluent or solvent.

The prior art also discloses plastic transfer sets for vials. Various vial transfer sets have been proposed for transferring fluid between a vial and a conventional syringe, wherein the transfer set is mounted on the vial for later use. The transfer set may include a piercing member, such as a needle cannula generally telescopically mounted in a tubular fluid transfer member mounted on the open end of the vial. Such transfer sets require few steps to reconstitute a dry material, but are not appropriate for all dry materials and delivery systems.

Dry materials available for reconstitution may also be stored directly in a syringe. For example, as described in U.S. Pat. No. 6,773,714, leuprolide acetate, a peptide drug, is lyophilized directly in a syringe prior to use. A biodegradable polymer and solvent solution is filled into another syringe. The two syringes are coupled together and the contents are drawn back and forth between the two syringes until the polymer/solvent solution and the leuprolide acetate are effectively mixed together, forming a flowable composition. The flowable composition is drawn into one syringe and the two syringes then disconnected. A needle is inserted onto the syringe containing the flowable composition and then injected through the needle into the body. Other flowable compositions are described in U.S. Pat. Nos. 5,324,519; 4,938,763; 5,702,716; 5,744,153; and 5,990,194.

Storage of a dry material in a syringe may also require only that a reconstitution fluid be added and available to be mixed and expelled in one step. One means of dispersing the liquid and excluding air from the syringe is to insert the liquid near the mouth and include a porous membrane or small aperture in the plunger; such membrane pores or aperture being sufficiently large to permit the passage of air but sufficiently small to prevent significant loss of the fluid pre or post reconstitution (U.S. Pat. App. Pub. No. 2003/0180262). Using this method, mixing is performed by pushing on the plunger, reducing the dead space and pushing the air near the plunger out, and pushing the dry mass into the wetted area near the mouth (holding the mouth up and capping so the liquid is not pushed out). There are disadvantages of this method, such that clogging of the bottom membrane makes the product unusable, and requires remanufacturing of the plunger specifications of an already accepted medical device, essentially producing a new device and potentially requiring regulatory approval.

### SUMMARY

An illustrative embodiment of the present invention relates to another means of reconstituting a dry material in a syringe, adding a reconstitution fluid, and excluding a significant amount of air in the syringe, by providing a means of inserting the fluid into the syringe as far away from the syringe mouth as possible. Then one may hold said syringe with its long axis vertical and its mouth pointing up and inject the reconstituting fluid into what is now the bottom of the cavity holding the dry material. As the syringe plunger is then pushed upward, the fluid and the originally dry contents of the syringe will also move upward and the air on top will be expelled. If further mixing is required, this can then be performed by passing the content back and forth between two syringes and significantly less foaming will occur since most of the air has already been expelled.

Provided herein is a novel device and method for easily inserting a reconstituting fluid into a syringe cavity farthest from the mouth of the syringe. The device consists of a suitable connector device to attach to the mouth of the syringe containing a material in need of reconstitution and a lumen (e.g., a tube, cannula, or hollow needle) that extends down through the mouth of that syringe into that part of the syringe cavity farthest from the mouth of said syringe. The connector device is also formed such that it has an aperture suitable to couple with the mouth of another syringe containing the fluid to be used in reconstituting the material in the first syringe.

In one embodiment, the invention provides methods of introducing fluid into a syringe that contains a material that is to be reconstituted. The material that is to be reconstituted may include an injectable solution, suspension, gel, paste, powder, or drug delivery system. In another embodiment, the present invention provides methods of adapting syringes that are commonly available in commerce for use as containers for materials that are to be reconstituted by addition of a reconstituting fluid.

In a further embodiment, the present invention provides a device for use with the described method of reconstituting filled syringes.

Accordingly, embodiments of the present invention remedy the deficiencies in the prior art and provide the following:

1. novel, specific mixing conditions that impart the desired efficiency of the drug delivery system; and.

2. a novel device to produce these mixing conditions through the use of an inverted lumen (e.g., a cannula), that imparts improved delivery efficiency compared to other known systems.

In an illustrative embodiment, the present invention provides a device (10) for reconstituting a material (12) to be reconstituted, comprising:

(a) a first structure (14) having at least one first inner portion (16) configured to receive at least a portion of a material (12) to be reconstituted, comprising a first proximal opening (18) and a first distal opening (20), wherein the first distal opening (20) has a first mating structure (22), and a first movable member (24) selectively inserted into said first inner portion (16) to urge out the material (12) to be reconstituted,

(b) a second structure (26) having at least one second inner portion (28) configured to receive at least a portion of a reconstituting material (29), comprising a second proximal opening (not shown) and a second distal opening (30), wherein the second distal opening (30) has a second mating structure (32), and a second movable member (not shown) selectively inserted into said second inner portion (28) to urge out the reconstituting material (29), and

(c) a connector device (34), comprising a first mating portion (36) configured to mate with the first mating structure (22), a second mating portion (38) configured to mate with the second mating structure (32), and a lumen (40) extending between the first (36) and second (38) mating portions and configured to permit communication between the materials in said first (14) and second (26) structures.

The device (10) may include at least one of the first (24) and second (not shown) movable members configured to substantially occupy, respectively, said at least one first (16) and second (28) inner portion. The first (22) and second (32) mating structures of the device (10) may include a male end and the first (36) and second (38) mating portions of the device may include a female end (as shown in FIG. 1A). The first (22) and second (32) mating structures of the device may also be female and the first (36) and second (38) mating portions of the device may be male (not shown).

At least one of the first (14) and second (26) structures may include a syringe. The device may include each of the first (14) and second (26) structures as a syringe, wherein the first (22) and second (32) mating structures may include luer tips (as shown in FIG. 1A), or the first (22) and second (32) mating structures may include luer lock tips ((42,44), as shown in FIG. 1C), or the first (22) and second (32) mating structures may include catheter tips (not shown), or the first (22) and second (32) mating structures may have a barbed tip (not shown). In embodiments wherein the first (22) and second (32) mating structures have a barbed tip, the first (36) and second (38) mating portions have an opening of appropriate diameter and geometry to mate with a barbed tip.

In another embodiment, as illustrated in FIG. 2, the present invention further provides a method of reconstituting a material (12) to be reconstituted, comprising:

(a) providing a first structure (14) having at least one first inner portion (16) configured to receive at least a portion of a material (12) to be reconstituted, comprising a first proximal opening (18) and a first distal opening (20), wherein the first distal opening (20) has a first mating structure (22), and a first movable member (24) selectively inserted into said first inner portion (16) to urge out the material (12) to be reconstituted,

(b) providing a connector device (34), comprising a first mating portion (36) configured to mate with the first mating structure (22), a second mating portion (38) configured to mate with a second mating structure (32), and a lumen (40) extending between the first (36) and second (38) mating portions and configured to permit communication between the materials in said first (14) and second (26) structures,

(c) connecting the first mating portion (36) to the first mating structure (22), thereby causing the lumen (40) to extend through the first mating structure (22) and into the first inner portion (16),

(d) providing a second structure (26) having at least one second inner portion (28) configured to receive at least a portion of a reconstituting material (29), comprising a second proximal opening (not shown) and a second distal opening (30), wherein the second distal opening (30) has a second mating structure (32), and a second movable member (not shown) selectively inserted into said second inner portion (28) to urge out the reconstituting material,

(e) connecting the second mating structure (32) to the second mating portion (38), and

(f) urging out reconstituting material (29) from the second inner portion (not shown) into the first structure (14) via said connector device (34), thereby mixing the reconstituting material (29) and the material (12) to be reconstituted.

Upon connecting the first mating portion (36) to the first mating structure (22), the first movable member (24) may be in substantial proximity to the lumen (40). The method may then involve disconnecting the first mating portion (36) from the first mating structure (22). At least one of the first (24) and second (not shown) movable members may be configured to substantially occupy, respectively, said at least one first (16) and second (28) inner portion. The first (22) and second (32) mating structures may include a male end and the first (36) and second (38) mating portions may include a female end. The first (22) and second (32) mating structures may also be female and the first (36) and second (38) mating portions may also be male.

At least one of the first (14) and second (26) structures may include a syringe. Each of the first (14) and second (26) structures may include a syringe, wherein the first (22) and second (32) mating structures may include luer tips, or the first (22) and second (32) mating structures may include luer lock tips (42, 44), or the first (22) and second (32) mating structures may include catheter tips, or the first (22) and second (32) mating structures may have a barbed tip. In embodiments wherein the first (22) and second (32) mating structures have a barbed tip, the first (36) and second (38) mating portions have an opening of appropriate diameter and geometry to mate with a barbed tip.

According to the described method, the material (12) to be reconstituted may include, e.g., injectable solutions, suspensions, gels, pastes, powders, and drug delivery systems. The material (12) to be reconstituted may also include, e.g., drugs, vaccines, and medicaments. In one embodiment, the material (12) to be reconstituted is a mixture of demineralized and/or ground bone particles. The reconstituting material (29) may include, e.g., sterile water, saline, solution containing antibiotic or other medication, whole blood, and serum. In one embodiment, the reconstituting material (29) is saline solution.

In one embodiment, the present invention further provides a method of reconstituting a material (12) to be reconstituted, comprising:

(a) providing a first structure (14) having at least one first inner portion (16) configured to receive at least a portion of a material (12) to be reconstituted, comprising: a first proximal opening (18) and a first distal opening (20), wherein the first distal opening (20) has a first mating structure (22), and a first movable member (24) selectively inserted into said inner portion (16) to urge out the material (12) to be reconstituted,

(b) providing a connector device (34), comprising: a first mating portion (36) configured to mate with the first mating structure (22), a second mating portion (38) configured to mate with a second mating structure (32), and a lumen (40) extending between the first (36) and second (38) mating portions and configured to permit communication between the materials in said first (14) and second (26) structures,

(c) connecting the first mating portion (36) to the first mating structure (22), thereby causing the lumen (40) to extend through the first mating portion (36) and into the first inner portion (16),

(d) inserting the material (12) to be reconstituted into the first inner portion (16),

(e) cooperatively connecting the first movable member (24) with the first structure (14),

(f) providing a second structure (26) having at least one second inner portion (28) configured to receive at least a portion of a reconstituting material (29), comprising a second proximal opening (not shown) and a second distal opening (30), wherein the second distal opening (30) has a second mating structure (32), and a second movable member (not shown) selectively inserted into said second inner portion (28) to urge out the reconstituting material (29),

(g) connecting the second mating structure (32) to the second mating portion (38), and

(h) urging out reconstituting material (29) from the second inner portion (28) into the first inner portion (16), thereby mixing the reconstituting material (29) and the material (12) to be reconstituted.

Upon connecting the first mating portion (36) to the first mating structure (22), the first movable member (24) may be in substantial proximity to the lumen (40). The method may further comprise disconnecting the first mating portion (36) from the first mating structure (22). At least one of the first (24) and second (not shown) movable members may be configured to substantially occupy, respectively, said at least one first (16) and second (28) inner portion. In one embodiment, the first (22) and second (32) mating structures include a male end, and the first (36) and second (38) mating portions include a female end. In another embodiment, the first (22) and second (32) mating structures may be female and the first (36) and second (38) mating portions are male.

In one embodiment, at least one of the first (14) and second (26) structures may include a syringe. In particular embodiments, each of the first (14) and second (26) structures may include a syringe, wherein the first (22) and second (32) mating structures may include luer tips, or the first (22) and second (32) mating structures may include luer lock tips (42, 44), or the first (22) and second (32) mating structures may include catheter tips, or the first (22) and second (32) mating structures may have a barbed tip. In embodiments wherein the first (22) and second (32) mating structures have a barbed tip, the first (36) and second (38) mating portions have an opening of appropriate diameter and geometry to mate with a barbed tip.

According to the described method, the material (12) to be reconstituted may include, e.g., injectable solutions, suspensions, gels, pastes, powders, and drug delivery systems. The material (12) to be reconstituted may also include, e.g., drugs, vaccines, and medicaments. In one embodiment, the material (12) to be reconstituted is a mixture of demineralized and/or ground bone particles. The reconstituting material (29) may include, e.g., sterile water, saline, solution containing antibiotic or other medication, whole blood, and serum. In one embodiment, the reconstituting material (29) is saline solution.

### BRIEF DESCRIPTION OF THE DRAWINGS

FIG. 1 shows general illustrations of the inverted cannula device including (A) the inverted cannula coupled to two syringes, with the reconstituting fluid syringe on top (partially depicted), the inverted cannula in the middle, and the mixing syringe at the bottom, (B) the inverted cannula separately, and (C) the inverted cannula between two luer lock syringes (partially depicted).

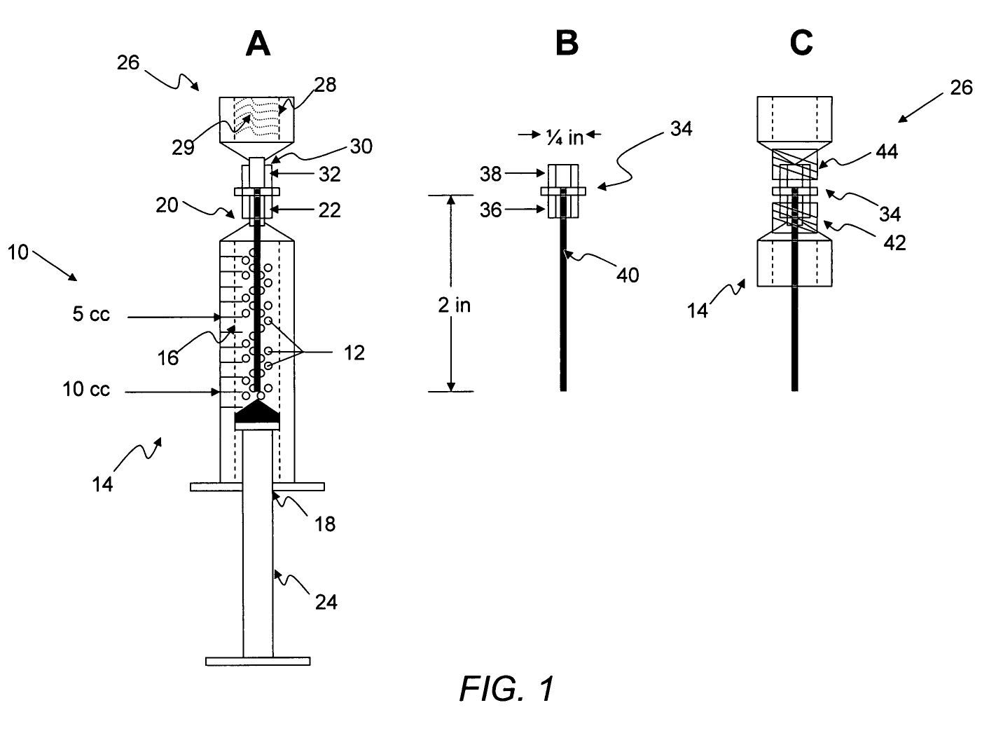

FIG. 2 shows another general illustration of the method of reconstituting a dry material including step (A) injecting fluid from the reconstituting fluid syringe through the inverted cannula into the bottom of the mixing syringe, (B) removing the reconstituting fluid syringe coupled to the inverted cannula from the mixing syringe, and (C) pushing the plunger of the mixing syringe up to allow the reconstituting fluid to mix with the dry material, without the fluid being forced out the mouth of the mixing syringe.

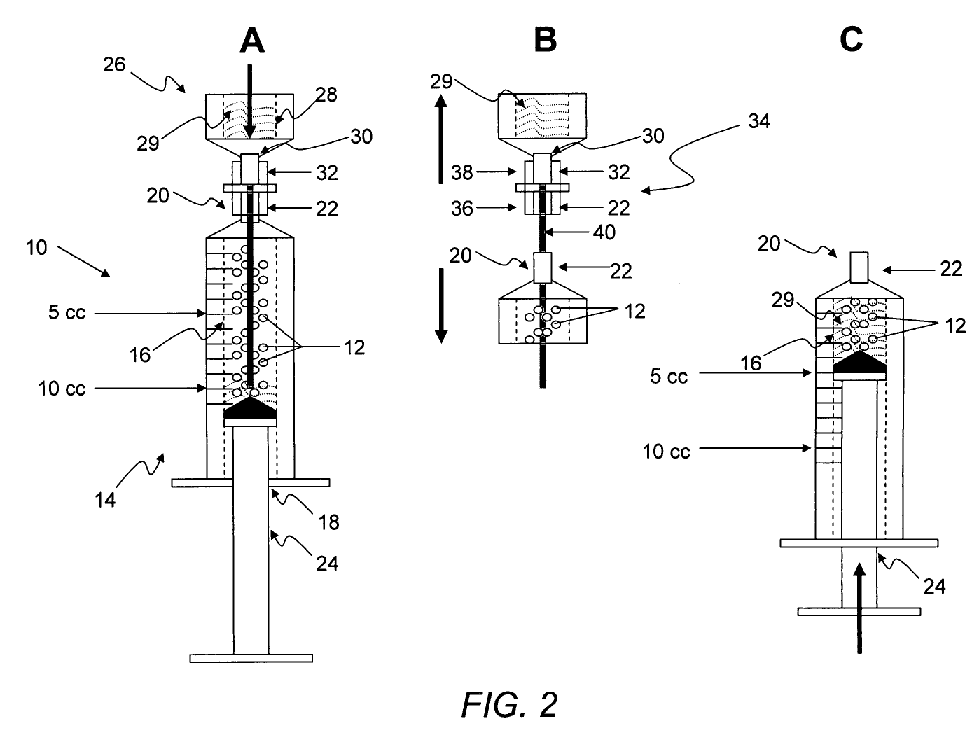

### DETAILED DESCRIPTION

In one aspect, the present invention relates to lumens, e.g., cannulas, for use with common syringes. For example, one illustrative embodiment of the device relates to a connector device (34) formed to have a female luer opening on one end (36) and a female luer opening on the other end (38). Inside the connector device may be affixed a narrow diameter lumen or tube (40) that may extend well beyond the first end (36) of the connector device. This connector device may then be mounted onto a syringe (14) with a male standard syringe tip or “luer” tip (22) such that the narrow tube (40) f the connector device traverses through the male luer tip of the syringe (14) and extends into the interior of the syringe (16). So arranged, dry powder or other material (12) to be reconstituted can be placed in the syringe (16) and the plunger (24) inserted until it impinges on the narrow tube (40) of the connector device (34). To reconstitute the material (12) in the mixing syringe (14), another syringe (26) with a male standard syringe tip or luer tip (32), that contains the reconstituting material (29) (e.g., a reconstituting fluid), may be attached to the other end (38) of the connector device (34), and the reconstituting material (29) may then be injected. In one aspect, holding vertical the mixing syringe (14) containing the material (12) to be reconstituted may facilitate the process, as gravity would pull the liquid away from the mouth end of the syringe. However, depending on the packing density and the hydrophilicity of the contents, holding the syringe (14) vertical is not always necessary because surface tension may be sufficient to keep the fluid near the end where it is injected. To complete the reconstitution process, the connector device (34) and its narrow tube (40) are removed from the syringe (14) and the plunger (24) pushed inward, expelling the air over the fluid/contents.

In a particular illustrative embodiment of the inverted cannula, both ends (36, 38) of the connector device (34) have female luer-lock openings, permitting a more secure attachment between the device (34) and the syringes (14, 26) used with it. In another embodiment, the connector device (34) is formed with a female luer or a female luer-lock opening on the end (38) that will couple with the fluid containing syringe (26) and formed with an opening to accept a male catheter tip syringe on the end (36) from which the narrow tube protrudes. This embodiment would be used, e.g., in instances where the reconstituted material has relatively high viscosity, such as, e.g., so-called bone pastes (a mixture of demineralized and/or ground bone particles in a water-based carrier such as gelatin). The reconstituted material will flow more easily through the larger diameter catheter tip than it would through a male luer tip.

In another embodiment, the connector device (34) is formed with a female luer or a female luer-lock opening on the end (38) that will couple with the reconstitution fluid syringe and with a barbed tip on the end (36) from which the narrow tube (40) protrudes. The barbed tip would be sized such that it will fit snugly into the opening of a male catheter tip.

While various embodiments of the present invention have been described above, it should be understood that they have been presented by way of example only, and not limitation. The present invention is not limited to the described embodiments, nor is it limited to a particular composition or material, nor is the present invention limited to a particular scale or size of production. Thus, the breadth and scope of the present invention should not be limited by any of the above-described exemplary embodiments, but should be defined only in accordance with the following claims and their equivalents.

### REFERENCES

1. Jansen, H. and Thibault, J., “Improved Transfer Set”, WO 01/28490, Filed Oct. 20, 1999. Published Apr. 26, 2001.

2. Dunn, R. L., Garrett, J. S., Ravivarapu, H., and Chandrashekar, B. L., “Polymeric delivery formulations of leuprolide with improved efficacy”, U.S. Pat. No. 6,773,714. Issued Aug. 10, 2004.

3. Wironen, J. F. Kao, P., and Bernhardt, A., “System for reconstituting pastes and methods of using same”, U.S. Pat. Appl. Pub. No. 2003/0180262. Filed Oct. 11, 2001. Published Sep. 25, 2003.

1. A device for reconstituting a material to be reconstituted, comprising:
(a) a first structure having at least one first inner portion configured to receive at least a portion of a material to be reconstituted, comprising:
a first proximal opening and a first distal opening, wherein the first distal opening has a first mating structure, and
a first movable member selectively inserted into said first inner portion to urge out the material to be reconstituted,
(b) a second structure having at least one second inner portion configured to receive at least a portion of a reconstituting material, comprising:
a second proximal opening and a second distal opening, wherein the second distal opening has a second mating structure, and
a second movable member selectively inserted into said second inner portion to urge out the reconstituting material, and
(c) a connector device, comprising:
a first mating portion configured to mate with the first mating structure,
a second mating portion configured to mate with the second mating structure, and
a lumen extending between the first and second mating portions and configured to permit communication between the materials in said first and second structures.

2. The device of claim 1, wherein at least one of the first and second movable members is configured to substantially occupy, respectively, said at least one first and second inner portion.

3. The device of claim 1, wherein the first and second mating structures are male and the first and second mating portions are female.

4. The device of claim 1, wherein the first and second mating structures are female and the first and second mating portions are male.

5. The device of claim 1, wherein at least one of the first and second structures is a syringe.

6. The device of claim 5, wherein each of the first and second structures is a syringe.

7. The device of claim 6, wherein the first and second mating structures are luer tips.

8. The device of claim 6, wherein the first and second mating structures are luer lock tips.

9. The device of claim 6, wherein the first and second mating structures are catheter tips.

10. The device of claim 6, wherein the first and second mating structures are a barbed tips.

11. A method of reconstituting a material to be reconstituted, comprising:
(a) providing a first structure having at least one first inner portion configured to receive at least a portion of a material to be reconstituted, comprising:
a first proximal opening and a first distal opening, wherein the first distal opening has a first mating structure, and
a first movable member selectively inserted into said first inner portion to urge out the material to be reconstituted,
(b) providing a connector device, comprising:
a first mating portion configured to mate with the first mating structure,
a second mating portion configured to mate with a second mating structure, and
a lumen extending between the first and second mating portions and configured to permit communication between the materials in said first and second structures,
(c) connecting the first mating portion to the first mating structure, thereby causing the lumen to extend through the first mating structure and into the first inner portion,
(d) providing a second structure having at least one second inner portion configured to receive at least a portion of a reconstituting material, comprising:
a second proximal opening and a second distal opening, wherein the second distal opening has a second mating structure, and
a second movable member selectively inserted into said second inner portion to urge out the reconstituting material,
(e) connecting the second mating structure to the second mating portion,
(f) urging out reconstituting material from the second inner portion into the first structure via said connector device, thereby mixing the reconstituting material and the material to be reconstituted.

12. The method of claim 11, wherein, upon connecting the first mating portion to the first mating structure, the first movable member is in substantial proximity to the lumen.

13. The method of claim 11, further comprising disconnecting the first mating portion from the first mating structure

14. The method of claim 11, wherein at least one of the first and second movable members is configured to substantially occupy, respectively, said at least one first and second inner portion.

15. The method of claim 11, wherein the first and second mating structures are male and the first and second mating portions are female.

16. The method of claim 11, wherein the first and second mating structures are female and the first and second mating portions are male.

17. The method of claim 11, wherein at least one of the first and second structures is a syringe.

18. The method of claim 17, wherein each of the first and second structures is a syringe.

19. The method of claim 18, wherein the first and second mating structures are luer tips.

20. The method of claim 18, wherein the first and second mating structures are luer lock tips.

21. The method of claim 18, wherein the first and second mating structures are catheter tips.

22. The method of claim 18, wherein the first and second mating structures are barbed tips.

23. The method of claim 11, wherein the material to be reconstituted is chosen from injectable solutions, suspensions, gels, pastes, powders, and drug delivery systems.

24. The method of claim 11, wherein the material to be reconstituted is chosen from drugs, vaccines, and medicaments.

25. The method of claim 11, wherein the material to be reconstituted is a mixture of demineralized and/or ground bone particles.

26. The method of claim 11, wherein the reconstituting material is chosen from sterile water, saline, solution containing antibiotic or other medication, whole blood, and serum.

27. The method of claim 11, wherein the reconstituting material is saline solution.

28. A method of reconstituting a material to be reconstituted, comprising:
(a) providing a first structure having at least one first inner portion configured to receive at least a portion of a material to be reconstituted, comprising:
a first proximal opening and a first distal opening, wherein the first distal opening has a first mating structure, and
a first movable member selectively inserted into said inner portion to urge out the material to be reconstituted,
(b) providing a connector device, comprising:
a first mating portion configured to mate with the first mating structure,
a second mating portion configured to mate with a second mating structure, and
a lumen extending between the first and second mating portions and configured to permit communication between the materials in said first and second structures,
(c) connecting the first mating portion to the first mating structure, thereby causing the lumen to extend through the first mating portion and into the first inner portion,
(d) inserting the material to be reconstituted into the first inner portion,
(e) cooperatively connecting the first movable member with the first structure,
(f) providing a second structure having at least one second inner portion configured to receive at least a portion of a reconstituting material, comprising:
a second proximal opening and a second distal opening, wherein the second distal opening has a second mating structure, and
a second movable member selectively inserted into said second inner portion to urge out the reconstituting material,
(g) connecting the second mating structure to the second mating portion,
(h) urging out reconstituting material from the second inner portion into the first inner portion, thereby mixing the reconstituting material and the material to be reconstituted.

29. The method of claim 28, wherein, upon connecting the first mating portion to the first mating structure, the first movable member is in substantial proximity to the lumen.

30. The method of claim 28, further comprising disconnecting the first mating portion from the first mating structure.

31. The method of claim 28, wherein at least one of the first and second movable members is configured to substantially occupy, respectively, said at least one first and second inner portion.

32. The method of claim 28, wherein the first and second mating structures are male and the first and second mating portions are female.

33. The method of claim 28, wherein the first and second mating structures are female and the first and second mating portions are male.

34. The method of claim 28, wherein at least one of the first and second structures is a syringe.

35. The method of claim 34, wherein each of the first and second structures is a syringe.

36. The method of claim 35, wherein the first and second mating structures are luer tips.

37. The method of claim 35, wherein the first and second mating structures are luer lock tips.

38. The method of claim 35, wherein the first and second mating structures are catheter tips.

39. The method of claim 35, wherein the first and second mating structures are barbed tips.

40. The method of claim 35, wherein the material to be reconstituted is chosen from injectable solutions, suspensions, gels, pastes, powders, and drug delivery systems.

41. The method of claim 35, wherein the material to be reconstituted is chosen from drugs, vaccines, and medicaments.

42. The method of claim 35, wherein the material to be reconstituted is a mixture of demineralized and/or ground bone particles.

43. The method of claim 35, wherein the reconstituting material is chosen from sterile water, saline, whole blood, and serum.

44. The method of claim 35, wherein the reconstituting material is saline solution.
